(19) United States
(12) Patent Application Publication
(10) Pub. No.: US 2007/0088019 A1
(43) Pub. Date: Apr. 19, 2007

(54) Macroheterocyclic compounds as kinase inhibitors

(76) Inventors: Han-Cheng Zhang, Lansdale, PA (US); Gee-Hong Kuo, Scotch Plains, NJ (US); Bruce E. Maryanoff, Forest Grove, PA (US); Hong Ye, Lansdale, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/528,092

(22) Filed: Sep. 27, 2006

Related U.S. Application Data
(60) Provisional application No. 60/722,072, filed on Sep. 29, 2005.

(51) Int. Cl. A61K 31/553; C07D 491/22

(52) U.S. Cl. 514/211.1; 540/489

(57) ABSTRACT

This invention is directed to macroheterocyclic compounds useful as kinase or dual-kinase inhibitors, methods for producing such compounds and methods for treating or ameliorating a kinase or dual-kinase mediated disease, condition or disorder.

### CROSS-REFERENCE TO RELATED APPLICATIONS

This present application claims benefit of U.S. Provisional Patent Application Ser. No. 60/722,072, filed Sep. 29, 2005, which is incorporated herein by reference in its entirety and for all purposes.

### FIELD OF THE INVENTION

This invention is directed to certain novel macroheterocyclic compounds, methods for preparing such compounds, and methods for treating or ameliorating a kinase or dual kinase mediated disease, condition or disorder. More particularly, this invention is directed to macrocyclic 1H-indole and 1H-pyrrolo[2,3-b]pyridine compounds useful as selective kinase or dual-kinase inhibitors, methods for producing such compounds and methods for treating or ameliorating a kinase or dual-kinase mediated disease, condition or disorder.

### BACKGROUND OF THE INVENTION

U.S. Pat. No. 5,624,949 to Heath, Jr., et. al., describes bis-indolemaleimide derivatives as PKC inhibitors and as selective PKCβ-I and PKCβ-II inhibitors, but does not disclose or suggest the compounds of the present invention.

U.S. Pat. No. 6,828,327 to Kuo et. al., describes macrocyclic compounds useful as kinase inhibitors, but does not disclose or suggest the compounds of the present invention.

### SUMMARY OF THE INVENTION

The present invention provides a macroheterocyclic compound of Formula (I): 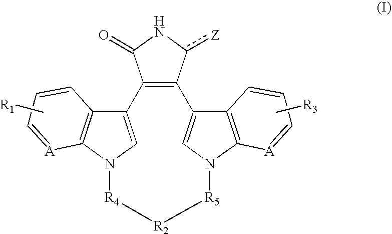
wherein
A is CH or N, whereby the A-containing ring system of Formula (I) thus forms 1H-indole or 1H-pyrrolo[2,3-b]pyridine, respectively;
Z is O, OH, or H, H;
R₁, and R₃ are independently selected from the group consisting of hydrogen, C₁₋₄alkyl, C₂₋₄alkenyl, C₂₋₄alkynyl, C₁₋₄alkoxy, C₁₋₄alkylthio, halogen, trifluoromethyl, trifluoromethoxy, hydroxy, hydroxy(C₁₋₄)alkyl, cyano, nitro, amino, and amino(C₁₋₄)alkyl; wherein amino and the amino portion of amino(C₁₋₄)alkyl are optionally and independently substituted with one to two C₁₋₄alkyl substituents;
R₄ and R₅ are independently C₂₋₈alkylene optionally substituted with oxo;
R₂ is —N(Rₐ)—C₁₋₄alkyl-X—C₁₋₄alkyl-N(R_{b})—, wherein C₁₋₄alkyl is optionally substituted with one to four substituents independently selected from the group consisting of C₁₋₄alkyl, C₂₋₄alkenyl, C₂₋₄alkynyl, C₁₋₄alkoxy, C₁₋₄alkoxy(C₁₋₄)alkyl, carboxyl, carboxyl(C₁₋₄)alkyl, C₁₋₄alkoxycarbonyl, C₁₋₄alkoxycarbonyl(C₁₋₄)alkyl, amino, amino(C₁₋₄)alkyl, halogen, (halo)₁₋₃(C₁₋₄)alkyl, (halo)₁₋₃(C₁₋₄)alkoxy, hydroxy, hydroxy(C₁₋₄)alkyl, and oxo; wherein amino and the amino portion of amino(C₁₋₄)alkyl are optionally and independently substituted with one to two C₁₋₄alkyl substituents;
Rₐ and R_{b} are independently selected from the group consisting of hydrogen, C₁₋₆alkyl, C₂₋₆alkenyl, and C₂₋₆alkynyl; wherein C₁₋₆alkyl, C₂₋₆alkenyl, and C₂₋₆alkynyl are optionally substituted with fluoro;
X is O or S;
such that the —R₄—R₂—R₅— containing macrocycle does not exceed 25 atoms in size;
and enantiomers, diastereomers, racemates, and pharmaceutically acceptable salts thereof.

The present invention is directed to macroheterocyclic compounds useful as a selective kinase or dual-kinase inhibitor. The present invention is further directed to compounds useful as inhibitors of kinases selected from the group consisting of protein kinase C and glycogen synthase kinase-3.

The present invention is further directed to compounds useful as selective inhibitors of kinases selected from the group consisting of protein kinase C α, protein kinase C β, protein kinase C γ, and glycogen synthase kinase-3β.

The present invention is also directed to methods for producing the instant macroheterocyclic compounds and pharmaceutical compositions and medicaments thereof.

The present invention is further directed to methods for treating or ameliorating a kinase or dual-kinase mediated disease, condition or disorder.

The method of the present invention is directed to treating or ameliorating a kinase mediated disease, condition or disorder such as, but not limited to, cardiovascular diseases, diabetes, diabetes-associated disorders, diabetic complications, inflammatory diseases, immunological disorders, dermatological disorders, oncological disorders, and CNS (central nervous system) disorders.

### DETAILED DESCRIPTION OF THE INVENTION

As used herein, with reference to substituents, the term “independently” means that when more than one of such substituent is possible, such substituents may be the same or different from each other.

As used herein, unless otherwise noted, “C₁₋₈alkyl” whether used alone or as part of a substituent group refers to straight and branched carbon chains having 1 to 8 carbon atoms or any number within this range, such as C₁₋₄alkyl. Therefore, designated numbers of carbon atoms (e.g. C₁₋₈) shall refer independently to the number of carbon atoms in an alkyl or cycloalkyl moiety or to the alkyl portion of a larger substituent in which alkyl appears as its prefix root. Fore example, C₁₋₆alkyl would include methyl, ethyl, propyl, butyl, pentyl and hexyl individually as well as sub-combinations thereof (e.g. C₁₋₂, C₁₋₃, C₁₋₄, C₁₋₅, C₂₋₆, C₃₋₆, C₄₋₆, C₅₋₆, C₂₋₅, etc.).

As used herein, unless otherwise noted, the term “C₁₋₈alkylene” refers to a biradical substituent formed from an alkyl group, as defined herein, in which the biradical is formed by the removal of two hydrogen atoms.

As used herein, unless otherwise noted, the term “C₁₋₈alkoxy” refers to an —O—(C₁₋₈)alkyl substituent group, wherein alkyl is as defined supra. Similarly, the terms “C₂₋₄alkenyl” and “C₂₋₄akynyl” refer to straight and branched carbon chains having 2 to 8 carbon atoms or any number within this range, wherein an alkenyl chain has at least one double bond in the chain and an alkynyl chain has at least one triple bond in the chain. An alkyl and alkoxy chain may be substituted on a carbon atom where allowed by available valences. In substituent groups with one or more alkyl groups such as (C₁₋₆alkyl)₂amino- the C₁₋₆alkyl groups of the dialkylamino may be the same or different.

The term “C₁₋₄alkylthio” refers to substituents of the formula: —S—(C₁₋₄)alkyl.

The term “C₁₋₄alkoxy(C₁₋₄)alkyl” refers to substituents of the formula: —(C₁₋₄)alkyl-(C₁₋₄)alkoxy.

The term “C₁₋₄alkoxycarbonyl” refers to substituents of the formula: —C(O)—O—(C₁₋₄)alkyl.

The term “C₁₋₄alkoxycarbonyl(C₁₋₄)alkyl” refers to substituents of the formula: —(C₁₋₄)alkyl-C(O)—O—(C₁₋₄)alkyl.

The term “amino” refers to substituents of the formula: —NH₂.

The term “amino(C₁₋₄)alkyl” refers to substituents of the formula: —(C₁₋₄)alkyl-NH₂.

The term “carboxyl” refers to substituents of the formula: —C(O)OH.

The term “carboxyl(C₁₋₄)alkyl” refers to substituents of the formula:

—(C₁₋₄)alkyl-C(O)—OH.

The terms “halogen” and “halo” refer to fluorine, chlorine, bromine and iodine.

The terms “(halo)₁₋₃(C₁₋₄)alkyl” and “(halo)₁₋₃(C₁₋₄)alkoxy” refer to substituents that are substituted with one or more halogen atoms in a manner that provides compounds which are stable and include those such as trifluoromethyl or trifluoromethoxy.

The term “hydroxy(C₁₋₄)alkyl” refers to substituents that are substituted with one or more hydroxy groups in a manner that provides compounds which are stable.

The [symbol] portion of [symbol] in the compounds of Formula (I) refers to one or two single bonds to Z, or a double bond to Z,
such that when Z is O, [symbol] is taken to form 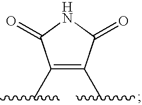
and such that when Z is OH, [symbol] is taken to form 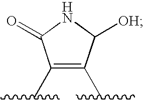
and such that when Z is H, H, [symbol] is taken to form

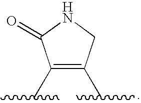

In general, under standard nomenclature rules (i.e. IUPAC) used throughout this disclosure, the terminal portion of the designated side chain is described first followed by the adjacent functionality toward the point of attachment. Thus, for example, a “phenylC₁-C₆alkylamidoC₁-C₆alkyl” substituent refers to a group of the formula:

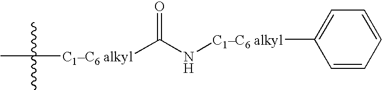

In certain instances, the following ring numbering convention was used to derive the nomenclature for compounds of the present invention: 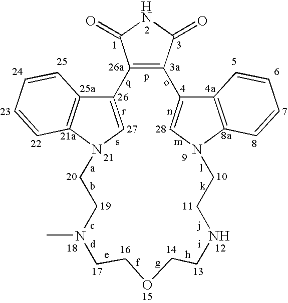
18-methyl-10,11,12,13,14,16,17,18,19,20-decahydro-15-oxa-2,9,12,18,21-pentaaza-1H-diindolo[1,2,3-m,n:3′,2′,1′-r,s]cyclononadec-14,16,18-trien[16,17-c]-pyrrole-2,5-dione (Compound 4)

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques known in the art as well as those methods set forth herein.

The term “subject” as used herein, refers to an animal, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease, condition or disorder being treated.

As used herein, the term “composition” is intended to encompass a product comprising the specified ingredients in the specified amounts, as well as any product which results, directly or indirectly, from combinations of the specified ingredients in the specified amounts.

Embodiments of the present invention include compounds of Formula (I) wherein:
(a). A is CH, whereby the A-containing ring system of Formula (I) thus forms 1H-indole;
(b). Z is O;
(c). R₁ and R₃ are independently selected from the group consisting of hydrogen, methyl, methoxy, halogen, and hydroxy;
(d). R₁ and R₃ are each hydrogen;
(e). R₄ and R₅ are each C₂₋₄alkylene;
(f). R₄ and R₅ are each ethylene;
(g). R₂ is —N(Rₐ)—C₁₋₄alkyl-X—C₁₋₄alkyl-N(R_{b})—;
(h). R₂ is —N(Rₐ)—CH₂CH₂—X—CH₂CH₂—N(R_{b})—;
(i). Rₐ and R_{b} are independently hydrogen or C₁₋₆alkyl;
(j). Rₐ and R_{b} are independently hydrogen or methyl;
(k). X is S; or
(I). X is O.

Further embodiments of the present invention are directed to compounds of Formula (Ia): 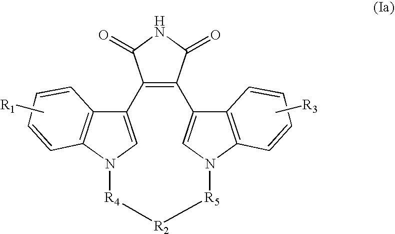
wherein
R₁, and R₃ are independently selected from the group consisting of hydrogen, C₁₋₄alkyl, C₂₋₄alkenyl, C₂₋₄alkynyl, C₁₋₄alkoxy, C₁₋₄alkylthio, halogen, trifluoromethyl, trifluoromethoxy, hydroxy, hydroxy(C₁₋₄)alkyl, cyano, nitro, amino, and amino(C₁₋₄)alkyl; wherein amino and the amino portion of amino(C₁₋₄)alkyl are optionally and independently substituted with one to two C₁₋₄alkyl substituents;
R₄ and R₅ are independently C₂₋₈ alkylene optionally substituted with oxo;
R₂ is —N(Rₐ)—C₁₋₄alkyl-X—C₁₋₄alkyl-N(R_{b})—, wherein C₁₋₄alkyl is optionally substituted with one to four substituents independently selected from the group consisting of C₁₋₄alkyl, C₂₋₄alkenyl, C₂₋₄alkynyl, C₁₋₄alkoxy, C₁₋₄alkoxy(C₁₋₄)alkyl, carboxyl, carboxyl(C₁₋₄)alkyl, C₁₋₄alkoxycarbonyl, C₁₋₄alkoxycarbonyl(C₁₋₄)alkyl, amino, amino(C₁₋₄)alkyl, halogen,
(halo)₁₋₃(C₁₋₄)alkyl, (halo)₁₋₃(C₁₋₄)alkoxy, hydroxy, hydroxy(C₁₋₄)alkyl, and oxo; wherein amino and the amino portion of amino(C₁₋₄)alkyl are optionally and independently substituted with one to two C₁₋₄alkyl substituents;
Rₐ and R_{b} are independently selected from the group consisting of hydrogen, C₁₋₆alkyl, C₂₋₆alkenyl, and C₂₋₆alkynyl; wherein C₁₋₆alkyl, C₂₋₆alkenyl, and C₂₋₆alkynyl are optionally substituted with fluoro;
X is O or S;
such that the —R₄—R₂—R₅— containing macrocycle does not exceed 25 atoms in size;
and enantiomers, diastereomers, racemates, and pharmaceutically acceptable salts thereof.

Another embodiment of the present invention is directed to compounds of Formula (Ia) wherein:
R₄ and R₅ are each C₂₋₄alkylene;
R₂ is —N(Rₐ)—C₁₋₄alkyl-X—C₁₋₄alkyl-N(R_{b})—;
Rₐ and R_{b} are independently hydrogen or C₁₋₄alkyl; and,

R₁ and R₃ are independently selected from the group consisting of hydrogen, methyl, methoxy, halogen, and hydroxy. Another embodiment of the present invention is directed to compounds of Formula (Ia) wherein Rₐ and R_{b} are independently hydrogen or methyl and R₁ and R₃ are independently selected from the group consisting of hydrogen, methyl, methoxy, halogen, and hydroxy.

Another embodiment of the present invention is directed to compounds of Formula (Ia) wherein R₁ and R₃ are each hydrogen; R₄ and R₅ are each ethylene; R₂ is —N(Rₐ)—CH₂CH₂—X—CH₂CH₂—N(R_{b})—; and, Rₐ and R_{b} are independently hydrogen or methyl.

Further embodiments of the present invention are directed to compounds of Formula (Ib): 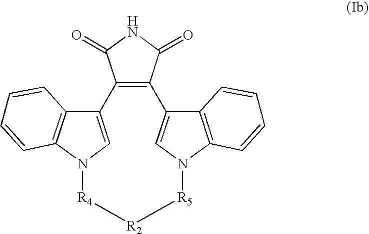
wherein
R₄ and R₅ are independently C₂₋₈alkylene optionally substituted with oxo;
R₂ is —N(Rₐ)—C₁₋₄alkyl-X—C₁₋₄alkyl-N(R_{b}), wherein C₁₋₄alkyl is optionally substituted with one to four substituents independently selected from the group consisting of C₁₋₄alkyl, C₂₋₄alkenyl, C₂₋₄alkynyl, C₁₋₄alkoxy, C₁₋₄alkoxy(C₁₋₄)alkyl, carboxyl, carboxyl(C₁₋₄)alkyl, C₁₋₄alkoxycarbonyl, C₁₋₄alkoxycarbonyl(C₁₋₄)alkyl, amino, amino(C₁₋₄)alkyl, halogen, (halo)₁₋₃(C₁₋₄)alkyl, (halo)₁₋₃(C₁₋₄)alkoxy, hydroxy, hydroxy(C₁₋₄)alkyl, and oxo; wherein amino and the amino portion of amino(C₁₋₄)alkyl are optionally and independently substituted with one to two C₁₋₄alkyl substituents;
Rₐ and R_{b} are independently selected from the group consisting of hydrogen, C₁₋₆alkyl, C₂₋₆alkenyl, and C₂₋₆alkynyl; wherein C₁₋₆alkyl, C₂₋₆alkenyl, and C₂₋₆alkynyl are optionally substituted with fluoro;
X is O or S;
such that the —R₄—R₂—R₅— containing macrocycle does not exceed 25 atoms in size;
and enantiomers, diastereomers, racemates, and pharmaceutically acceptable salts thereof.

Another embodiment of the present invention is directed to compounds of Formula (Ib), wherein:
R₄ and R₅ are each C₂₋₄alkylene; and,
Rₐ and R_{b} are independently hydrogen or C₁₋₄alkyl.

Another embodiment of the present invention is directed to compounds of Formula (Ib) wherein:
R₄ and R₅ are each ethylene;
R₂ is-N(Rₐ)—CH₂CH₂—X—CH₂CH₂—N(R_{b})—; and,
Rₐ and R_{b} are independently hydrogen or methyl.

An embodiment of the present invention includes compounds of Formula (I) selected from the group consisting of:

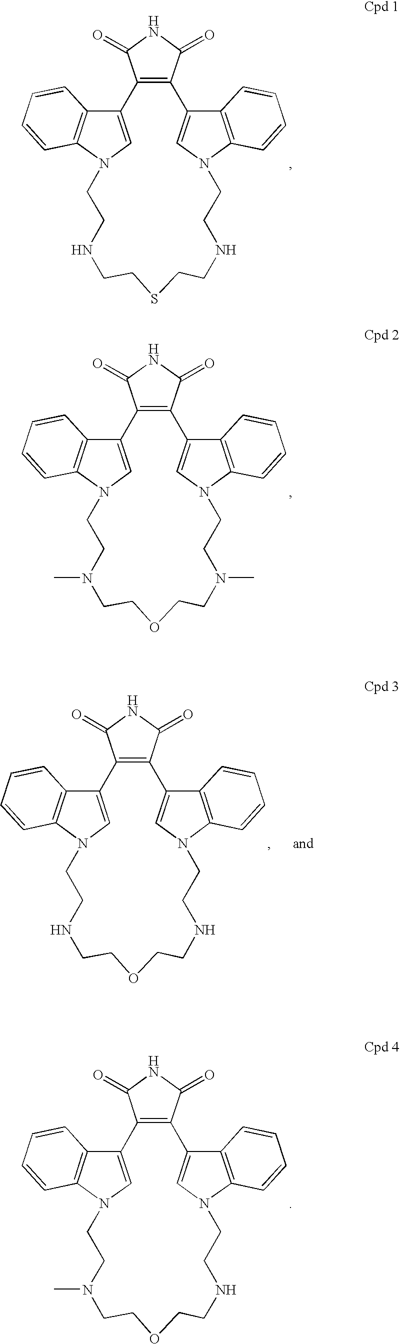

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids and bases which may be used in the preparation of pharmaceutically acceptable salts include the following: acids including acetic acid, 2,2-dichloroactic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hippuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid; and bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

The present invention includes within its scope prodrugs of the compounds of this invention. In general, such prodrugs will be functional derivatives of the compounds that are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

Where the processes for the preparation of the compounds according to the invention give rise to a mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-d-tartaric acid and/or (+)-di-p-toluoyl-l-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

Even though the compounds of the present invention (including their pharmaceutically acceptable salts and pharmaceutically acceptable solvates) can be administered alone, they will generally be administered in admixture with a pharmaceutical carrier, excipient or diluent selected with regard to the intended route of administration and standard pharmaceutical or veterinary practice. Thus, the present invention is directed to pharmaceutical and veterinary compositions comprising compounds of Formula (I) and one or more pharmaceutically acceptable carriers, excipients or diluents.

By way of example, in the pharmaceutical and veterinary compositions of the present invention, the compounds of the present invention may be admixed with any suitable binder(s), lubricant(s), suspending agent(s), coating agent(s), and/or solubilising agent(s).

Tablets or capsules of the compounds may be administered singly or two or more at a time, as appropriate. It is also possible to administer the compounds in sustained release formulations.

Alternatively, the compounds of the general Formula (I) can be administered by inhalation or in the form of a suppository or pessary, or they may be applied topically in the form of a lotion, solution, cream, ointment or dusting powder. An alternative means of transdermal administration is by use of a skin patch. For example, they can be incorporated into a cream consisting of an aqueous emulsion of polyethylene glycols or liquid paraffin. They can also be incorporated, at a concentration of between 1 and 10% by weight, into an ointment consisting of a white wax or white soft paraffin base together with such stabilizers and preservatives as may be required.

For some applications, preferably the compositions are administered orally in the form of tablets containing excipients such as starch or lactose, or in capsules or ovules either alone or in admixture with excipients, or in the form of elixirs, solutions or suspensions containing flavoring or coloring agents.

The compositions (as well as the compounds alone) can also be injected parenterally, for example intracavernosally, intravenously, intramuscularly or subcutaneously. In this case, the compositions will comprise a suitable carrier or diluent.

For parenteral administration, the compositions are best used in the form of a sterile aqueous solution which may contain other substances, for example enough salts or monosaccharides to make the solution isotonic with blood.

For buccal or sublingual administration the compositions may be administered in the form of tablets or lozenges which can be formulated in a conventional manner.

By way of further example, pharmaceutical and veterinary compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate the major site of absorption. For parenteral administration, the carrier will usually consist of sterile water and other ingredients may be added to increase solubility or preservation. Injectable suspensions or solutions may also be prepared utilizing aqueous carriers along with appropriate additives.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those skilled in that art. To be administered in the form of a transdermal delivery system, the dosage administration will, of course, be continuous rather than intermittent throughout the dosage regimen.

It is also apparent to one skilled in the art that the therapeutically effective dose for active compounds of the invention or a pharmaceutical composition thereof will vary according to the desired effect. Therefore, optimal dosages to be administered may be readily determined by one skilled in the art and will vary with the particular compound used, the mode of administration, the strength of the preparation, and the advancement of the disease condition. In addition, factors associated with the particular subject being treated, including subject age, weight, diet and time of administration, will result in the need to adjust the dose to an appropriate therapeutic level. The above dosages are thus exemplary of the average case. There can, of course, be individual instances where higher or lower dosage ranges are merited, and such are within the scope of this invention.

The invention also provides a pharmaceutical or veterinary pack or kit comprising one or more containers filled with one or more of the ingredients of the pharmaceutical and veterinary compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

A therapeutically effective amount for use of the instant compounds or a pharmaceutical composition thereof comprises a dose range of from about 0.001 mg/kg/day to about 300 mg/kg/day, from about 0.01 mg/kg/day to about 150 mg/kg/day, from about 0.5 mg/kg/day to about 5.0 mg/kg/day or, from about 1.0 mg/kg/day to about 3.0 mg/kg/day of active ingredient in a regimen of about 1 to 4 times per day for an average (70 kg) human; although, it is apparent to one skilled in the art that the therapeutically effective amount for active compounds of the invention will vary as will the conditions being treated.

Protein Kinase C Isoforms

Protein kinase C (PKC) is known to play a key role in intracellular signal transduction (cell-cell signaling), gene expression and in the control of cell differentiation and growth. The PKC family is composed of twelve isoforms that are further classified into 3 subfamilies: the calcium dependent classical PKC isoforms alpha (α), beta-I (β-I), beta-II (β-II) and gamma (γ); the calcium independent PKC isoforms delta (δ), epsilon (ε), eta (η), theta (θ) and mu (μ); and, the atypical PKC isoforms zeta (ζ), lambda (λ) and iota (ι).

Certain disease states tend to be associated with elevation of particular PKC isoforms. The PKC isoforms exhibit distinct tissue distribution, subcellular localization and activation-dependent cofactors. For example, the α and β isoforms of PKC are selectively induced in vascular cells stimulated with agonists such as vascular endothelial growth factor (VEGF) (P. Xia, et al., J. Clin. Invest., 1996, 98, 2018) and have been implicated in cellular growth, differentiation, and vascular permeability (H. Ishii, et al., J. Mol. Med., 1998, 76, 21). The elevated blood glucose levels found in diabetes leads to an isoform-specific elevation of the β-II isoform in vascular tissues (Inoguchi, et al., Proc. Natl. Acad. Sci. USA, 1992, 89, 11059-11065). A diabetes-linked elevation of the β isoform in human platelets has been correlated with the altered response of the platelets to agonists (Bastyr III, E. J. and Lu, J., Diabetes, 1993, 42, (Suppl. 1) 97A). The human vitamin D receptor has been shown to be selectively phosphorylated by PKCβ. This phosphorylation has been linked to alterations in the functioning of the receptor (Hsieh, et al., Proc. Natl. Acad. Sci. USA, 1991, 88, 9315-9319; Hsieh, et al., J. Biol. Chem., 1993, 268, 15118-15126). In addition, the work has shown that the β-II isoform is responsible for erythroleukemia cell proliferation while the α isoform is involved in megakaryocyte differentiation in these same cells (Murray, et al., J. Biol. Chem., 1993, 268, 15847-15853).

Cardiovascular Diseases

PKC activity plays an important role in cardiovascular diseases. Increased PKC activity in the vasculature has been shown to cause increased vasoconstriction and hypertension (Bilder, G. E., et al., J. Pharmacol. Exp. Ther., 1990, 252, 526-530). PKC inhibitors block agonist-induced smooth muscle cell proliferation (Matsumoto, H. and Sasaki, Y., Biochem. Biophys. Res. Commun., 1989,158,105-109). PKC β triggers events leading to the induction of Egr-1 (Early Growth Factor-1) and tissue factor under hypoxic conditions (as part of the oxygen deprivation-mediated pathway for triggering procoagulant events) (Yan, S-F, et al., J. Biol. Chem., 2000, 275, 16, 11921-11928). PKC β is suggested as a mediator for production of PAI-1 (Plasminogen Activator Inhibitor-1) and is implicated in the development of thrombosis and atherosclerosis (Ren, S, et al., Am. J. Physiol., 2000, 278, (4, Pt. 1), E656-E662). PKC inhibitors are useful in treating cardiovascular ischemia and improving cardiac function following ischemia (Muid, R. E., et al., FEBS Lett., 1990, 293, 169-172; Sonoki, H. et al., Kokyu-To Junkan, 1989, 37, 669-674). Elevated PKC levels have been correlated with an increase in platelet function in response to agonists (Bastyr III, E. J. and Lu, J., Diabetes, 1993, 42, (Suppl. 1)97A). PKC has been implicated in the biochemical pathway in the platelet-activating factor (PAF) modulation of microvascular permeability (Kobayashi, et al., Amer. Phys. Soc., 1994, H1214-H1220). PKC inhibitors affect agonist-induced aggregation in platelets (Toullec, D., et al., J. Biol. Chem., 1991, 266, 15771-15781). Accordingly, PKC inhibitors may be indicated for use in treating cardiovascular disease, ischemia, thrombotic conditions, atherosclerosis and restenosis.

Diabetes

Excessive activity of PKC has been linked to insulin signaling defects and therefore to the insulin resistance seen in Type II diabetes (Karasik, A., et al., J. Biol. Chem., 1990, 265, 10226-10231; Chen, K. S., et al., Trans. Assoc. Am. Physicians, 1991, 104, 206-212; Chin, J. E., et al., J. Biol. Chem., 1993, 268, 6338-6347).

Diabetes-Associated Disorders

Studies have demonstrated an increase in PKC activity in tissues known to be susceptible to diabetic complications when exposed to hyperglycemic conditions (Lee, T-S., et al., J. Clin. Invest., 1989, 83, 90-94; Lee, T-S., et al., Proc. Natl. Acad. Sci. USA, 1989, 86, 5141-5145; Craven, P. A. and DeRubertis, F. R., J. Clin. Invest., 1989, 87, 1667-1675; Wolf, B. A., et al., J. Clin. Invest., 1991, 87, 31-38; Tesfamariam, B., et al., J. Clin. Invest., 1991, 87, 1643-1648). For example, activation of the PKC-β-II isoform plays an important role in diabetic vascular complications such as retinopathy (Ishii, H., et al., Science, 1996, 272, 728-731) and PKCβ has been implicated in development of the cardiac hypertrophy associated with heart failure (X. Gu, et al., Circ. Res., 1994, 75, 926; R. H. Strasser, et al., Circulation, 1996, 94,1551). Overexpression of cardiac PKCβII in transgenic mice caused cardiomyopathy involving hypertrophy, fibrosis and decreased left ventricular function (H. Wakasaki, et al., Proc. Natl. Acad. Sci. USA, 1997, 94, 9320).

Inflammatorv Diseases

PKC inhibitors block inflammatory responses such as the neutrophil oxidative burst, CD3 down-regulation in T-lymphocytes and phorbol-induced paw edema (Twoemy, B., et al., Biochem. Biophys. Res. Commun., 1990,171,1087-1092; Mulqueen, M. J., et al. Agents Actions, 1992, 37, 85-89). PKC β has an essential role in the degranulation of bone marrow-derived mast cells, thus affecting cell capacity to produce IL-6 (Interleukin-6) (Nechushtan, H., et al., Blood ,2000 (March), 95, 5, 1752-1757). PKC plays a role in enhanced ASM (Airway Smooth Muscle) cell growth in rat models of two potential risks for asthma: hyperresponsiveness to contractile agonists and to growth stimuli (Ren, S, et al., Am. J. Physiol., 2000, 278, (4, Pt. 1), E656-E662). PKC β-1 overexpression augments an increase in endothelial permeability, suggesting an important function in the regulation of the endothelial barrier (Nagpala, P. G., et al., J. Cell Physiol., 1996, 2, 249-55). PKC β mediates activation of neutrophil NADPH oxidase by PMA and by stimulation of Fcy receptors in neutrophils (Dekker, L. V., et al., Biochem. J, 2000, 347, 285-289). Thus, PKC inhibitors may be indicated for use in treating inflammation and asthma.

Immunological Disorders

PKC may be useful in treating or ameliorating certain immunological disorders. While one study suggests that HCMV (Human Cytomegalovirus) inhibition is not correlated with PKC inhibition (Slater, M. J., et al., Bioorg. & Med. Chem., 1999, 7, 1067-1074), another study showed that the PKC signal transduction pathway synergistically interacted with the cAMP-dependent PKA pathway to activate or increase HIV-1 transcription and viral replication and was abrogated with a PKC inhibitor (Rabbi, M. F., et al., Virology, 1998 (Jun. 5), 245, 2, 257-69). Therefore, an immunological disorder may be treated or ameliorated as a function of the affected underlying pathway's response to up- or down-regulation of PKC.

PKC β deficiency also results in an immunodeficiency characterized by impaired humoral immune responses and a reduced B cell response, similar to X-linked immunodeficiency in mice and plays an important role in antigen receptor-mediated signal transduction (Leitges, M., et al., Science (Wash., D.C.), 1996, 273, 5276, 788-789). Accordingly, transplant tissue rejection may be ameliorated or prevented by suppressing the immune response using a PKC β inhibitor.

Dermatological Disorders

Abnormal activity of PKC has been linked to dermatological disorders characterized by abnormal proliferation of keratinocytes, such as psoriasis (Horn, F., et al., J. Invest. Dermatol., 1987, 88, 220-222; Raynaud, F. and Evain-Brion, D., Br. J. Dermatol., 1991, 124, 542-546). PKC inhibitors have been shown to inhibit keratinocyte proliferation in a dose-dependent manner (Hegemann, L., et al., Arch. Dermatol. Res., 1991, 283,456-460; Bollag, W. B., et al., J. Invest. Dermatol., 1993,100, 240-246).

Oncological Disorders

PKC activity has been associated with cell growth, tumor promotion, uncontrolled cell growth and cancer (Rotenberg, S. A. and Weinstein, I. B., Biochem. Mol. Aspects Sel. Cancer, 1991, 1, 25-73; Ahmad, et al., Molecular Pharmacology, 1993, 43, 858-862). PKC inhibitors are known to be effective in preventing tumor growth in animals (Meyer, T., et al., Int. J. Cancer, 1989, 43, 851-856; Akinagaka, S., et al., Cancer Res., 1991, 51, 4888-4892). PKC β-1 and β-2 expression in differentiated HD3 colon carcinoma cells blocked their differentiation, enabling them to proliferate in response to basic FGF (Fibroblast Growth Factor) like undifferentiated cells, increasing their growth rate and activating several MBP (Myelin-Basic Protein) kinases, including p57 MAP (Mitogen-Activated Protein) kinase (Sauma, S., et al., Cell Growth Differ., 1996, 7, 5, 587-94). PKC a inhibitors, having an additive therapeutic effect in combination with other anti-cancer agents, inhibited the growth of lymphocytic leukemia cells (Konig, A., et al., Blood, 1997, 90, 10, Suppl. 1 Pt. 2). PKC inhibitors enhanced MMC (Mitomycin-C) induced apoptosis in a time-dependent fashion in a gastric cancer cell-line, potentially indicating use as agents for chemotherapy-induced apoptosis (Danso, D., et al., Proc. Am. Assoc. Cancer Res., 1997, 38, 88 Meet., 92). Therefore, PKC inhibitors may be indicated for use in ameliorating cell and tumor growth, in treating or ameliorating cancers (such as leukemia or colon cancer) and as adjuncts to chemotherapy.

PKC α (by enhancing cell migration) may mediate some proangiogenic effects of PKC activation while PKC δ may direct antiangiogenic effects of overall PKC activation (by inhibiting cell growth and proliferation) in capillary endothelial cells, thus regulating endothelial proliferation and angiogenesis (Harrington, E. O., et al., J. Biol. Chem., 1997, 272, 11, 7390-7397). PKC inhibitors inhibit cell growth and induce apoptosis in human glioblastoma cell lines, inhibit the growth of human astrocytoma xenografts and act as radiation sensitizers in glioblastoma cell lines (Begemann, M., et al., Anticancer Res. (Greece), 1998 (Jul.-Aug.), 18, 4A, 2275-82). PKC inhibitors, in combination with other anti-cancer agents, are radiation and chemosensitizers useful in cancer therapy (Teicher, B. A., et al., Proc. Am. Assoc. Cancer Res., 1998, 39, 89 Meet., 384). PKC β inhibitors (by blocking the MAP kinase signal transduction pathways for VEGF (Vascular Endothelial Growth Factor) and bFGF (basic Fibrinogen Growth Factor) in endothelial cells), in a combination regimen with other anti-cancer agents, have an anti-angiogenic and antitumor effect in a human T98G glioblastoma multiforme xenograft model (Teicher, B. A., et al., Clinical Cancer Research, 2001 (March), 7, 634-640). Accordingly, PKC inhibitors may be indicated for use in ameliorating angiogenesis and in treating or ameliorating cancers (such as breast, brain, kidney, bladder, ovarian or colon cancers) and as adjuncts to chemotherapy and radiation therapy.

Central Nervous System Disorders

PKC activity plays a central role in the functioning of the CNS (Huang, K. P., Trends Neurosci., 1989, 12, 425-432) and PKC is implicated in Alzheimer's disease (Shimohama, S., et al., Neurology, 1993, 43, 1407-1413) and inhibitors have been shown to prevent the damage seen in focal and central ischemic brain injury and brain edema (Hara, H., et al., J. Cereb. Blood Flow Metab., 1990, 10, 646-653; Shibata, S., et al., Brain Res., 1992, 594, 290-294). Accordingly, PKC inhibitors may be indicated for use in treating Alzheimers disease and in treating neurotraumatic and ischemia-related diseases.

The long-term increase in PKC γ (as a component of the phosphoinositide 2^{nd} messenger system) and muscarinic acetylcholine receptor expression in an amygdala-kindled rat model has been associated with epilepsy, serving as a basis for the rat's permanent state of hyperexcitability (Beldhuis, H. J. A., et al., Neuroscience, 1993, 55, 4, 965-73). Therefore, PKC inhibitors may be indicated for use in treating epilepsy.

The subcellular changes in content of the PKC γ and PKC β-II isoenzymes for animals in an in-vivo thermal hyperalgesia model suggests that peripheral nerve injury contributes to the development of persistent pain (Miletic, V., et al., Neurosci. Lett., 2000, 288, 3, 199-202). Mice lacking PKCγ display normal responses to acute pain stimuli, but almost completely fail to develop a neuropathic pain syndrome after partial sciatic nerve section (Chen, C., et al., Science (Wash., D. C.), 1997, 278, 5336, 279-283). PKC modulation may thus be indicated for use in treating chronic pain and neuropathic pain.

PKC has demonstrated a role in the pathology of conditions such as, but not limited to, cardiovascular diseases, diabetes, diabetes-associated disorders, inflammatory diseases, immunological disorders, dermatological disorders, oncological disorders and central nervous system disorders.

Glycogen Synthase Kinase-3

Glycogen synthase kinase-3 (GSK-3) is a serine/threonine protein kinase composed of two isoforms (α and β ) which are encoded by distinct genes. GSK-3 is one of several protein kinases which phosphorylate glycogen synthase (GS) (Embi, et al., Eur. J. Biochem, 1980, 107, 519-527). The α and β isoforms have a monomeric structure of 49 and 47 kD respectively and are both found in mammalian cells. Both isoforms phosphorylate muscle glycogen synthase (Cross, et al., Biochemical Journal, 1994, 303, 21-26) and these two isoforms show good homology between species (human and rabbit GSK-3α are 96% identical).

Diabetes

Type II diabetes (or Non-Insulin Dependent Diabetes Mellitus, NIDDM) is a multifactorial disease. Hyperglycemia is due to insulin resistance in the liver, muscle and other tissues coupled with inadequate or defective secretion of insulin from pancreatic islets. Skeletal muscle is the major site for insulin-stimulated glucose uptake. In this tissue, glucose removed from the circulation is either metabolised through glycolysis and the TCA (tricarboxylic acid) cycle or stored as glycogen. Muscle glycogen deposition plays the more important role in glucose homeostasis and Type II diabetic subjects have defective muscle glycogen storage. The stimulation of glycogen synthesis by insulin in skeletal muscle results from the dephosphorylation and activation of glycogen synthase (Villar-Palasi C. and Lamer J., Biochim. Biophys. Acta, 1960, 39, 171-173, Parker P. J., et al., Eur. J. Biochem., 1983,130, 227-234, and Cohen P., Biochem. Soc. Trans., 1993, 21, 555-567). The phosphorylation and dephosphorylation of GS are mediated by specific kinases and phosphatases. GSK-3 is responsible for phosphorylation and deactivation of GS, while glycogen bound protein phosphatase 1 (PP1 G) dephosphorylates and activates GS. Insulin both inactivates GSK-3 and activates PP1 G (Srivastava A. K. and Pandey S. K., Mol. and Cellular Biochem., 1998, 182, 135-141).

Studies suggest that an increase in GSK-3 activity might be important in Type II diabetic muscle (Chen, et al., Diabetes, 1994, 43, 1234-1241). Overexpression of GSK-30 and constitutively active GSK-3β (S9A, S9e) mutants in HEK-293 cells resulted in suppression of glycogen synthase activity (Eldar-Finkelman, et al., PNAS, 1996, 93, 10228-10233) and overexpression of GSK-3β in CHO cells, expressing both insulin receptor and insulin receptor substrate 1 (IRS-1) resulted in impairment of insulin action (Eldar-Finkelman and Krebs, PNAS, 1997, 94, 9660-9664). Recent evidence for the involvement of elevated GSK-3 activity and the development of insulin resistance and Type II diabetes in adipose tissue has emerged from studies undertaken in diabetes and obesity prone C57BL/6J mice (Eldar-Finkelman, et al., Diabetes, 1999, 48, 1662-1666).

Inflammatory Diseases

Studies on fibroblasts from the GSK-3β knockout mouse indicate that inhibition of GSK-3 may be useful in treating inflammatory disorders or diseases through the negative regulation of NFkB activity (Hoeflich K. P., et al., Nature, 2000, 406, 86-90).

Dermatological Disorders

The finding that transient β-catenin stabilization may play a role in hair development (Gat, et al., Cell, 1998, 95, 605-614) suggests that GSK-3 inhibitors could also be used in the treatment of baldness.

Central Nervous System Disorders

In addition to modulation of glycogen synthase activity, GSK-3 also plays an important role in the CNS disorders. GSK-3 inhibitors may be of value as neuroprotectants in the treatment of acute stroke and other neurotraumatic injuries (Pap and Cooper, J. Biol. Chem., 1998, 273, 19929-19932). Lithium, a low mM inhibitor of GSK-3, has been shown to protect cerebellar granule neurons from death (D′Mello, et al., Exp. Cell Res., 1994, 211, 332-338) and chronic lithium treatment has demonstrable efficacy in the middle cerebral artery occlusion model of stroke in rodents (Nonaka and Chuang, Neuroreport, 1998, 9(9), 2081-2084).

Tau and β-catenin, two known in vivo substrates of GSK-3, are of direct relevance in consideration of further aspects of the value of GSK-3 inhibitors in relation to treatment of chronic neurodegenerative conditions. Tau hyperphosphorylation is an early event in neurodegenerative conditions such as Alzheimer's disease and is postulated to promote microtubule disassembly. Lithium has been reported to reduce the phosphorylation of tau, enhance the binding of tau to microtubules and promote microtubule assembly through direct and reversible inhibition of GSK-3 (Hong M. et al J. Biol. Chem., 1997, 272(40), 25326-32). β-catenin is phosphorylated by GSK-3 as part of a tripartite axin protein complex resulting in β-catenin degradation (Ikeda, et al., EMBO J., 1998, 17, 1371-1384). Inhibition of GSK-3 activity is involved in the stabilization of catenin and promotes β-catenin-LEF-1/TCF transcriptional activity (Eastman, GrosschedI, Curr. Opin. Cell Biol., 1999, 11, 233). Studies have also suggested that GSK-3 inhibitors may also be of value in the treatment of schizophrenia (Cotter D., et al. Neuroreport, 1998, 9, 1379-1383; Lijam N., et al., Cell, 1997, 90, 895-905) and manic depression (Manji, et al., J. Clin. Psychiatry, 1999, 60, (Suppl 2)27-39 for review).

Accordingly, compounds found useful as GSK-3 inhibitors could have further therapeutic utility in the treatment of diabetes, inflammatory diseases, dermatological disorders and central nervous system disorders.

An embodiment of the present invention is a method for treating or ameliorating a selective kinase or dual-kinase mediated disease, condition or disorder in a subject in need thereof comprising administering to the subject a therapeutically effective amount of an instant compound or pharmaceutical composition thereof.

Another embodiment of the present invention is a method for treating or ameliorating diabetes or Alzheimer's disease in a subject in need thereof comprising administering to the subject a therapeutically effective amount of an instant compound or pharmaceutical composition thereof.

Another embodiment of the present invention is use of the compound of Formula (I) in the manufacture of a medicament for treating or ameliorating a kinase or dual-kinase mediated disease, condition or disorder.

The therapeutically effective amount of the compounds of Formula (I) exemplified in such a method is from about 0.001 mg/kg/day to about 300 mg/kg/day, from about 0.01 mg/kg/day to about 150 mg/kg/day, from about 0.5 to about 5.0 mg/kg/day or, from about 1.0 to about 3.0 mg/kg/day. The compounds may be administered on a regimen of 1 to 4 times per day.

Embodiments of the present invention include the use of a compound of Formula (I) for the preparation of a medicament for treating or ameliorating a kinase or dual-kinase mediated disease, condition or disorder in a subject in need thereof.

A further embodiment of the present invention includes the use of a composition comprising a compound of Formula (I) for the preparation of a medicament for treating or ameliorating a disorder selected from the group consisting of cardiovascular diseases, diabetes, diabetes-associated disorders, inflammatory diseases, immunological disorders, dermatological disorders, oncological disorders and CNS (central nervous system) disorders.

The present invention includes a method for treating a kinase or dual-kinase mediated disease, condition or disorder. More particularly, the present invention includes a method for inhibiting kinases selected from protein kinase C or glycogen synthase kinase-3; and, even more particularly, a kinase selected from the group consisting of protein kinase C α, protein kinase C β (such as β-I or β-II), protein kinase C γ, and glycogen synthase kinase-3β.

The term “dual-kinase” refers to the inhibitory activity of compounds of the present invention against one or more of the aforementioned kinases.

An embodiment of the present invention is a method for treating a kinase or dual-kinase mediated disease, condition or disorder including, but not limited to, cardiovascular diseases, diabetes, diabetes-associated disorders, inflammatory diseases, immunological disorders, dermatological disorders, oncological disorders and CNS (central nervous system) disorders.

Embodiments of the present invention include a compound or pharmaceutical composition thereof advantageously co-administered in combination with other agents for treating, reducing or ameliorating the effects of a kinase or dual-kinase mediated disease, condition or disorder.

The term “dual-kinase mediated disease, condition or disorder” refers to diseases, conditions or disorders mediated by one or more of the aforementioned kinases.

For example, in the treatment of diabetes, especially Type II diabetes, a compound of Formula (I) or pharmaceutical composition thereof may be used in combination with other agents, especially insulin or antidiabetic agents including, but not limited to, insulin secretagogues (such as sulphonylureas), insulin sensitizers including, but not limited to, glitazone insulin sensitizers (such as thiazolidinediones), biguamides or a glucosidase inhibitors.

The combination product is a product that comprises the co-administration of a compound of Formula (I) or a pharmaceutical composition thereof and an additional agent for treating or ameliorating a kinase or dual-kinase mediated disorder, or for treating a disorder selected from the group consisting of cardiovascular diseases, diabetes, diabetes-associated disorders, inflammatory diseases, immunological disorders, dermatological disorders, oncological disorders and CNS disorders.

The term combination product further comprises a product that is sequentially administered where the product comprises a compound of Formula (I) or pharmaceutical composition thereof and an additional agent, administration of a pharmaceutical composition containing a compound of Formula (I) or pharmaceutical composition thereof and an additional agent or the essentially simultaneous administration of a separate pharmaceutical composition containing a compound of Formula (I) or pharmaceutical composition thereof and a separate pharmaceutical composition containing an additional agent.

Additionally, the compounds of the present invention may further be administered in combination with a sulfamate compound of formula (I) as disclosed in Maryanoff et al., U.S. Pat. No. 4,513,006, which is hereby incorporated by reference, in its entirety. A particularly preferred sulfamate compound disclosed in Maryanoff et al., in U.S. Pat. No. 4,513,006 is topiramate, also known by its chemical name 2,3:4,5-di-O-isopropylidene-(β)-D-fructopyranose sulfamate, a compound of the following structure:

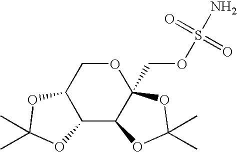

The sulfamate compounds of formula (I) as disclosed in Maryanoff et al., U.S. Pat. No. 4,513,006 are useful in treating, preventing and/or preventing the progression of various disorders and diseases, including, but not limited to (a) epilepsy and related disorders; (b) diabetes, Syndrome X, impaired oral glucose tolerance and other metabolic disorders; (c) elevated blood pressure; (d) elevated lipid levels; (e) obesity and overweight condition, as would be recognized by one skilled in the art.

Preferably, one or more of the compounds of the present invention are administered in combination with topiramate for the treatment of a disorder selected from the group consisting of cardiovascular diseases, diabetes, diabetes-associated disorders, inflammatory diseases, immunological disorders, dermatological disorders, oncological disorders and CNS disorders.

Preferably, the topiramate is administered in an amount in the range of from about 10 to about 400 mg per day, more preferably from about 25 to about 250 mg per day, more preferably from about 25 to about 200 mg per day.

The ubiquitous nature of the PKC and GSK isoforms and their important roles in physiology provide incentive to produce highly selective PKC and GSK inhibitors. Given the evidence demonstrating linkage of certain isoforms to disease states, it is reasonable to assume that inhibitory compounds that are selective to one or two PKC isoforms or to a GSK isoform relative to the other PKC and GSK isoforms and other protein kinases are superior therapeutic agents. Such compounds should demonstrate greater efficacy and lower toxicity by virtue of their specificity. Accordingly, it will be appreciated by one skilled in the art that a particular compound of Formula (I) is selected where it is therapeutically effective for a particular kinase or dual-kinase mediated disorder based on the modulation of the disorder through the demonstration of selective kinase or dual-kinase inhibition in response to that compound. Experiments exemplifying selective kinase or dual-kinase inhibition are provided in the examples. The usefulness of a compound of Formula (I) as a selective kinase or dual-kinase inhibitor can be determined according to the methods disclosed herein and based on the data obtained to date, it is anticipated that a particular compound will be useful in inhibiting one or more kinase or dual-kinase mediated disorders and therefore is useful in one or more kinase or dual-kinase mediated disorders.

Therefore, the term “kinase or dual-kinase mediated disorders” as used herein, includes, and is not limited to, cardiovascular diseases, diabetes, diabetes-associated disorders, inflammatory diseases, immunological disorders, dermatological disorders, oncological disorders and CNS disorders.

Cardiovascular diseases include, and are not limited to, acute stroke, heart failure, cardiovascular ischemia and impaired cardiac function following ischemia, thrombosis, atherosclerosis, hypertension, restenosis, retinopathy of prematurity or age-related macular degeneration.

Diabetes includes insulin dependent diabetes or Type II non-insulin dependent diabetes mellitus. Diabetes-associated disorders include, and are not limited to, impaired glucose tolerance, insulin signaling defects, insulin resistance, metabolic syndrome X, diabetic retinopathy, proliferative retinopathy, retinal vein occlusion, macular edema, cardiac hypertrophy associated with heart failure, cardiomyopathy, nephropathy or neuropathy.

Inflammatory diseases include, and are not limited to, neutrophil and cytokine migration, bone marrow degranulation, vascular permeability, inflammation, asthma, rheumatoid arthritis or osteoarthritis.

Immunological disorders include, and are not limited to, transplant tissue rejection, HIV-1 transcription and viral replication or immunological disorders treated or ameliorated by PKC modulation.

Dermatological disorders include, and are not limited to, psoriasis, hair loss or baldness.

Oncological disorders include, and are not limited to, cancer or tumor growth (such as breast, brain, kidney, bladder, ovarian or colon cancer or lymphocytic leukemia) and other diseases associated with uncontrolled cell proliferation such as recurring benign tumors as well as including proliferative angiopathy and angiogenesis; and, includes use for compounds of Formula (I) as an adjunct to chemotherapy and radiation therapy.

CNS disorders include, and are not limited to, chronic pain, neuropathic pain, epilepsy, chronic neurodegenerative conditions (such as dementia or Alzheimer's disease), mood disorders (such as schizophrenia, manic depression or neurotraumatic, cognitive decline) and ischemia-related diseases (as a result of head trauma (from acute ischemic stroke, injury or surgery) or transient ischemic stroke (from coronary bypass surgery or other transient ischemic conditions)).

Another embodiment of the present invention is a method of treating a disorder selected from the group consisting of cardiovascular diseases, diabetes, diabetes-associated disorders, inflammatory diseases, immunological disorders, dermatological disorders, oncological disorders and CNS disorders comprising administering to a subject in need thereof a compound of Formula (I) or a pharmaceutical composition of the present invention.

A representative compound of Formula (I) or a form thereof for use in the therapeutic methods and pharmaceutical compositions, medicines or medicaments described herein includes a compound selected from the group consisting of:

| Cpd | Name |
|---|---|
| 1 | 10,11,12,13,14,16,17,18,19,20-decahydro-15-thio-2,9,12,18,21- |
| | pentaaza-1H-diindolo[1,2,3-m,n:3′,2′,1′-r,s]cyclononadec-14,16,18- |
| | trien[16,17-c]-pyrrole-2,5-dione, |
| 2 | 12,18-dimethyl-10,11,12,13,14,16,17,18,19,20-decahydro-15-oxa- |
| | 2,9,12,18,21-pentaaza-1H-diindolo[1,2,3-m,n:3′,2′,1′- |
| | r,s]cyclononadec-14,16,18-trien[16,17-c]-pyrrole-2,5-dione, |
| 3 | 10,11,12,13,14,16,17,18,19,20-decahydro-15-oxa-2,9,12,18,21- |
| | pentaaza-1H-diindolo[1,2,3-m,n:3′,2′,1′-r,s]cyclononadec-14,16,18- |
| | trien[16,17-c]-pyrrole-2,5-dione, and |
| 4 | 18-methyl-10,11,12,13,14,16,17,18,19,20-decahydro-15-oxa- |
| | 2,9,12,18,21-pentaaza-1H-diindolo[1,2,3-m,n:3′,2′,1′- |
| | r,s]cyclononadec-14,16,18-trien[16,17-c]-pyrrole-2,5-dione. |

General Synthetic Methods

Representative compounds of the present invention can be synthesized in accordance with the general synthetic methods described below and are illustrated in the schemes that follow. Since the schemes are an illustration, the invention should not be construed as being limited by the chemical reactions and conditions expressed. The preparation of the various starting materials used in the schemes is well within the skill of persons versed in the art.

Abbreviations used in the instant specification, particularly the Schemes and Examples, are as follows:

| Abbreviation | Meaning |
|---|---|
| BSA | Bovine Serum Albumin |
| DIPEA or DIEA | diisopropylethylamine |
| DMF | N,N-dimethylformamide |
| DMSO | dimethylsulfoxide |
| DTT | dithiothreitol |
| EGTA | ethylene glycol-bis(beta-aminoethylether)- |
| | N,N,N′,N′-tetraacetic acid |
| EtOAc | ethyl acetate |
| HEPES | 4-(2-hydroxyethyl)-1-piperazine ethane |
| | sulfonic acid |
| HPLC | High Pressure Liquid Chromatography |
| MeOH | methanol |
| Ms₂O | methanesulfonic anhydride |
| NT | Not tested |
| Py | pyridine |
| TBDMS | tetrabutyldimethylsilyl |
| TCA | trichloroacetic acid |
| THF | tetrahydrofuran |
| TLC | Thin Layer Chromatography |
| Tris HCl | tris-(hydroxymethyl)-aminomethyl |
| | hydrochloride |

Scheme A describes the preparation of certain intermediates and compounds of the present invention wherein R₄ and R₅ are C₂₋₈alkyl. The compounds of formula A1 and formula A4 are commercially available materials or may be made by those skilled in the art using conventional methods and known materials.

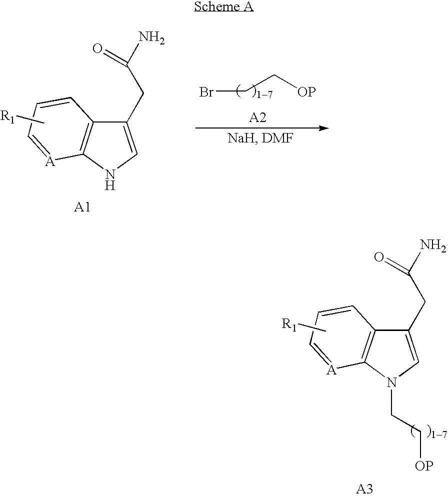

A compound of formula A1 may be alkylated under basic conditions with a hydroxyl-protected compound of formula A2 via nucleophilic displacement, wherein P is a suitable hydroxyl protecting group, such as TBDMS, to afford a compound of formula A3.

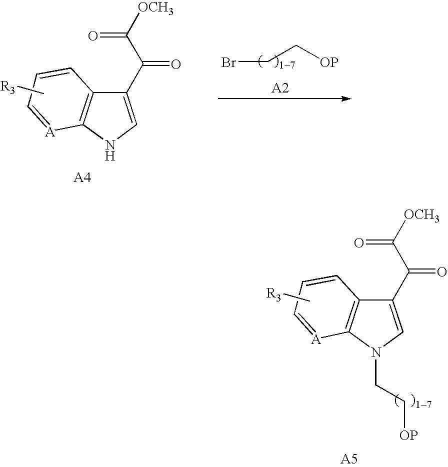

Similarly, a compound of formula A4 may be alkylated with a compound of formula A2 in a solvent such as DMF, in the presence of a base such as cesium carbonate, to afford a compound of formula A5.

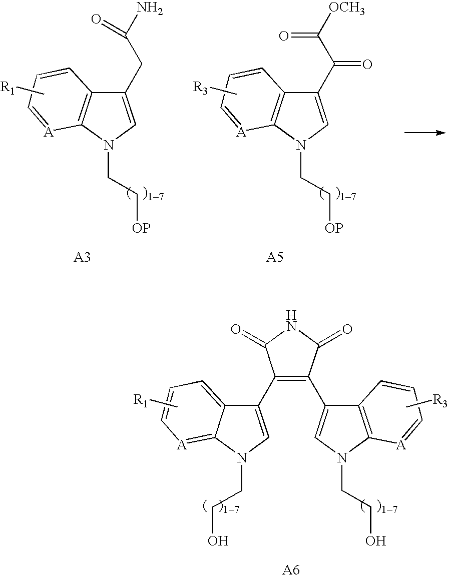

Condensation of a compound of formula A3 with a compound of formula A5 under basic conditions affords the maleimide portion of a compound of formula A6. Subsequent to the condensation, an acidic deprotection of the hydroxyl protecting groups, P, affords the bis-hydroxyl compound of formula A6.

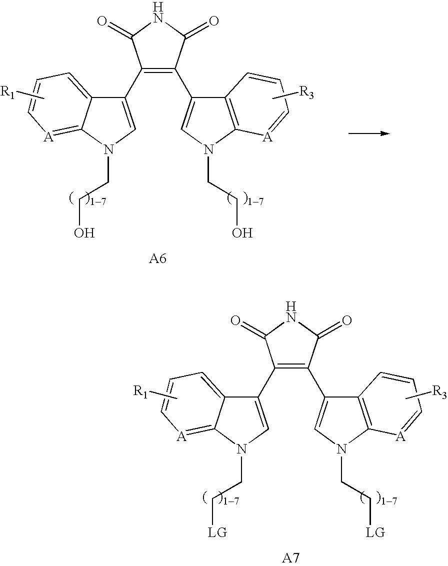

The bis-hydroxyl groups may each be converted to an appropriate leaving group (LG) using conventional chemical reagents and methods known to one skilled in the art to provide a compound of formula A7. Suitable leaving groups include halides, mesylates, triflates, or the like.

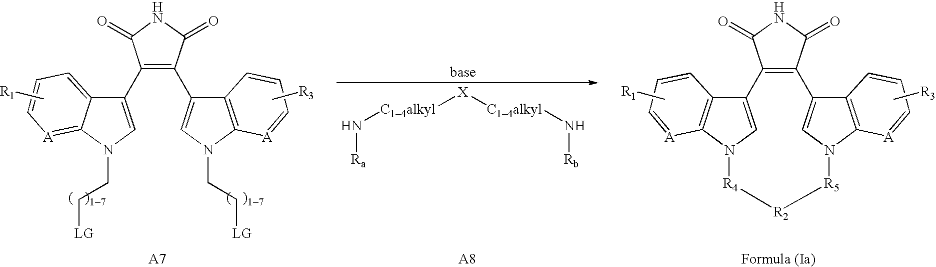

A compound of formula A7 may be condensed with a compound of formula A8 via a double nucleophilic displacement to form the macrocyclic compound of Formula (Ia), wherein the C₁₋₈alkyl portions of Compound A7 and the chain of Compound A8 are incorporated into —R₄—R₂—R₅— containing macrocycle of the Compound of Formula (Ia).

To prepare compounds of the present invention wherein one of Rₐ and R_{b} is hydrogen and the other of Rₐ and R_{b} is C₁₋₄alkyl, a compound of Formula (Ia) wherein Rₐ and R_{b} are both hydrogen may be treated with an alkylating agent, such as an alkyl halide, to form a mixture of mono and bis alkylated products. The products may then be separated by conventional separation techniques. For the purposes of the present invention, preparative thin layer chromatography was employed.

Compounds of the present invention of Formula (I), wherein Z is (H, H), may be prepared via the treatment of a compound of Formula (Ia) (wherein Z is O) with an appropriate reducing agent such as lithium aluminum hydride or Zn—Hg under anhydrous conditions. One skilled in the art will recognize that when a compound of Formula (I) is asymmetric, treatment with a reducing agent may result in a mixture of reduction products. Therefore, a separation of regioisomers may be required to isolate the individual products. Separation techniques known to those skilled in the art include recrystallization and/or chromatography.

Similarly, compounds of the present invention of Formula (I), wherein Z is OH, may be prepared via the treatment of a compound of Formula (Ia) (wherein Z is O) with a reducing agent such as lithium aluminum hydride. Separation techniques as described above may be employed for the separation of mixtures of regioisomers.

Specific Synthetic Methods

Specific compounds which are representative of this invention were prepared as per the following examples and reaction sequences; the examples and the diagrams depicting the reaction sequences are offered by way of illustration, to aid in the understanding of the invention and should not be construed to limit in any way the invention set forth in the claims which follow thereafter. The depicted intermediates may also be used in subsequent examples to produce additional compounds of the present invention. No attempt has been made to optimize the yields obtained in any of the reactions. One skilled in the art would know how to increase such yields through routine variations in reaction times, temperatures, solvents and/or reagents.

All chemicals were obtained from commercial suppliers and used without further purification. ¹H and ¹³C NMR spectra were recorded on a Bruker AC 300B (300 MHz proton) or a Bruker AM-400 (400 MHz proton) spectrometer with Me₄Si as an internal standard (s=singlet, d=doublet, t=triplet, br=broad). APCI-MS and ES-MS were recorded on a VG Platform II mass spectrometer; methane was used for chemical ionization, unless noted otherwise. Accurate mass measurements were obtained by using a VG ZAB 2-SE spectrometer in the FAB mode. TLC was performed with Whatman 250-μm silica gel plates. Preparative TLC was performed with Analtech 1000-μm silica gel GF plates. Flash column chromatography was conducted with flash column silica gel (40-63 μm) and column chromatography was conducted with standard silica gel. HPLC separations were carried out on three Waters PrepPak® Cartridges (25×100 mm, Bondapak® C 18, 15-20 μm, 125 Å) connected in series; detection was at 254 nm on a Waters 486 UV detector. Analytical HPLC was carried out on a Supelcosil ABZ+PLUS column (5 cm×2.1 mm), with detection at 254 nm on a Hewlett Packard 1100 UV detector. Microanalysis was performed by Robertson Microlit Laboratories, Inc.

### EXAMPLE 1

### 10,11,12,13,14,16,17,18,19,20-decahydro-15-thio-2,9,12,18,21-pentaaza-1H-diindolo[1,2,3-m,n:3′,2′,1′-r,s]cyclononadec-14,16,18-trien[16,17-c]-pyrrole-2,5-dione (Compound 1)

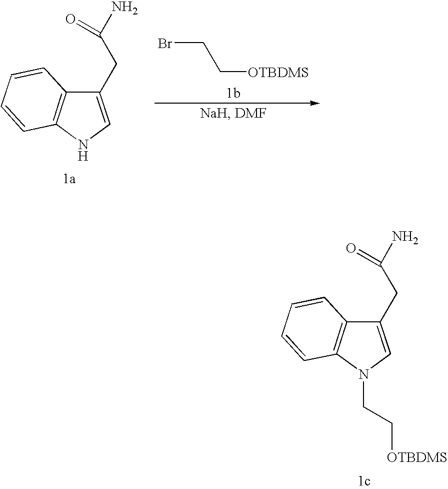

A mixture of Compound 1a (3.50 g, 20 mmol), Compound 1b (5.29 g, 22 mmol) and 60% NaH (0.88 g, 22 mmol) in DMF (350 mL) was stirred at 0° C. for 30 min and then r.t overnight. The reaction was quenched by slowly addition of water under ice bath. The mixture was extracted with EtOAc several times. The combined extracts were sequentially washed with water and brine and then dried (Na₂SO₄), evaporated in vacuo. The residue was separated by flash column chromatography (CH₂Cl₂/MeOH, 98:2) to give Compound 1c as a viscous oil. ¹H NMR (CDCl₃) δ 7.69 (d, J=7.9 Hz, 1H), 7.47 (d, J=8.2 Hz, 1H), 7.34 (m, 1H), 7.26 (m, 2H), 5.77 (bs, 1H), 5.62 (bs, 1H), 4.34 (t, J=5.5 Hz, 2H), 4.03 (t, J=5.5 Hz, 2H), 3.84 (s, 2H), 0.94 (s, 9H), 0.1 (s, 6H). ES-MS m/z 333 (MH⁺).

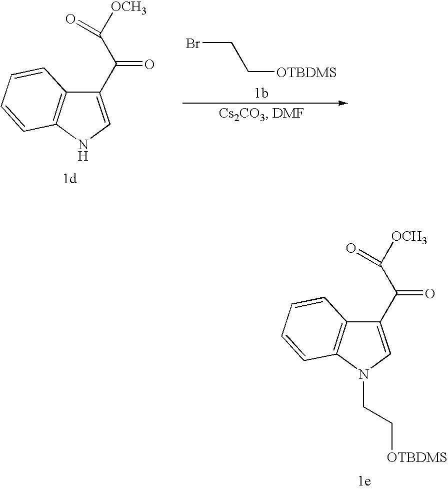

A mixture of Compound 1d (4.00 g, 19.7 mmol), Compound 1b (5.19 g, 21.7 mmol) and cesium carbonate (7.06 g, 21.7 mmol) in DMF (40 mL) was stirred at 50° C. for 4 h and then filtered. The filtrate was evaporated in vacuo and the residue was separated by flash column chromatography (EtOAc/heptane, 1:2) to give Compound 1e as a viscous oil. ¹HNMR (CDCl₃) δ 8.61 (m, 2H), 7.51 (m, 3H), 4.45 (t, J=5.1 Hz, 2H), 4.13 (m, 2H), 4.10 (s, 3H), 0.96 (s, 9H), 0.1 (s, 6H). ES-MS m/z 362 (MH⁺).

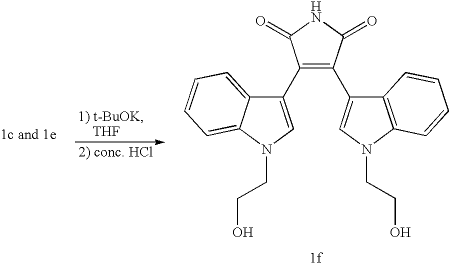

A volume of 1.0 M potassium t-butoxide in THF (28 mL, 28 mmol) was added dropwise to a suspension of the ester Compound 1e (2.8 g, 7.8 mmol) and the amide Compound 1c (1.83 g, 5.5 mmol) in dry THF (20 mL) under Argon that had been cooled to 0° C. The resulting mixture was stirred at 0° C. for 1 h and room temperature for 1 h, then concentrated HCl (10 mL) was added and the mixture was again stirred at room temperature for another 30 min. The mixture was partitioned between EtOAc and H₂O, the two layers were separated and the aqueous layer was extracted with EtOAc (4×50 mL). The combined extracts were sequentially washed with water, saturated aq. NaHCO₃ and brine and then dried (Na₂SO₄) and evaporated in vacuo. The crude mixture was purified by flash chromatography to give Compound 1f as an orange solid. ¹H NMR (DMSO) δ 7.78 (s, 2H), 7.48 (d, J=8.2 Hz, 2H), 7.03 (t, J=7.5 Hz, 2H), 6.83 (d, J=7.9 Hz, 2H), 6.68 (t, J=7.4 Hz, 2H), 4.92 (t, J=5.2 Hz, 2H), 4.26 (t, J=5.5 Hz, 4H), 3.68 (m, 4H). ES-MS m/z 416 (MH⁺).

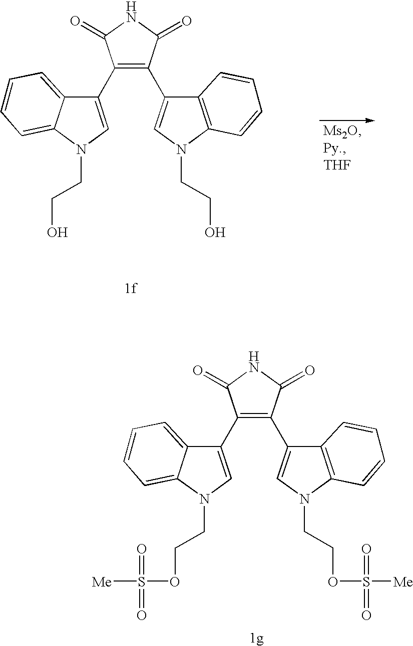

A portion of Ms₂O (methanesulfonic anhydride) (340 mg, 1.96 mmol) was added to a solution of Compound 1f (204 mg, 0.49 mmol) and Py (pyridine) (233 mg, 2.95 mmol) in THF (10 mL). The reaction was stirred at 50° C. for 3 h and then the reaction mixture was cooled to room temperature. A portion of THF (10 mL) and 1.0 N aq. HCl (20 mL) were added and the mixture was stirred at room temperature for 10 min, then extracted with EtOAc (200 mL). The organic phase was sequentially washed with 1.0 N aq. HCl (30 mL), water and brine, and then dried (Na₂SO₄) and evaporated in vacuo to give Compound 1g as a dark red-orange solid. ¹H NMR (CDCl₃) δ 7.72 (s, 2H), 7.30 (d, J=8.3 Hz, 2H), 7.13 (t, J=7.6 Hz, 2H), 6.97 (d, J=8.0 Hz, 2H), 6.79 (t, J=7.5 Hz, 2H), 4.50 (m, 8H), 2.82 (s, 6H). ES-MS m/z 572.3 (MH⁺).

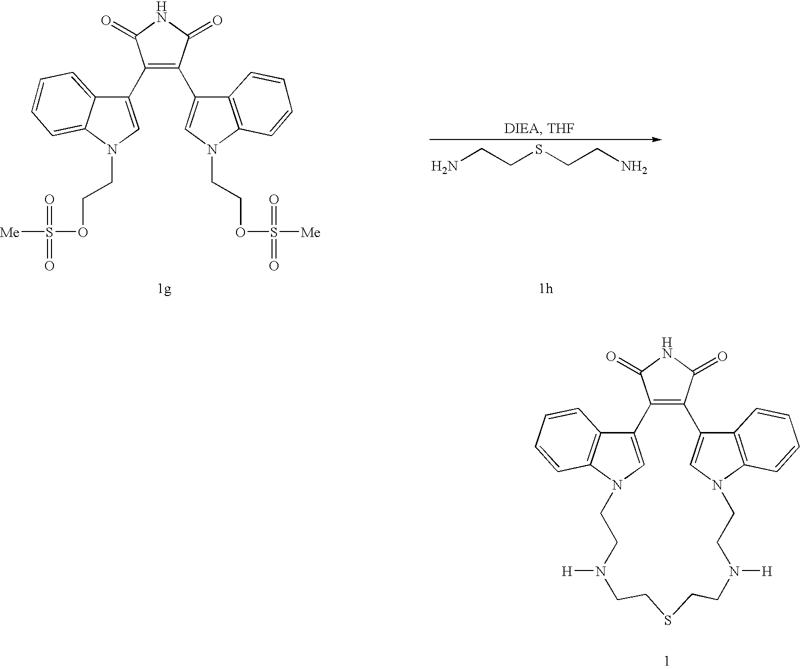

To a solution of Compound 1g (148 mg, 0.26 mmol) in DMF (15 mL) was added 2-(2-amino-ethylsulfanyl)-ethylamine (0.125 g, 1.04 mmol, 1h) and DIEA (200 mg, 1.55 mmol). The mixture was heated to 100° C. for 7 h. The mixture was extracted with EtOAc several times, washed with brine, dried (Na₂SO₄) and concentrated to give a red oil. The crude mixture was purified by preparative TLC (CH₂Cl₂ /MeOH/NH₄OH 95/5/0.5 ) to give Compound 1 as an orange solid. ¹H NMR (300 MHz, CDCl₃) δ 7.60 (s, 2H), 7.41 (d, J=8.2 Hz, 2H), 7.23 (m, 4H), 7.00 (t, J=7.5 Hz, 2H), 4.19 (m, 4H), 2.96 (m, 4H), 2.59 (m, 4H), 2.36 (m, 4H). FAB-HRMS calcd for C₂₈H₂₉N₅O₂S+H⁺, 500.2120, Found, 500.2119.

### EXAMPLE 2

### 12,18-dimethyl-10,11,12,13,14,16,17,18,19,20-decahydro-15-oxa-2,9,12,18,21-pentaaza-1H-diindolo[1,2,3-m,n:3′,2′,1′-r,s]cyclononadec-14,16,18-trien[16,17-c]-pyrrole-2,5-dione

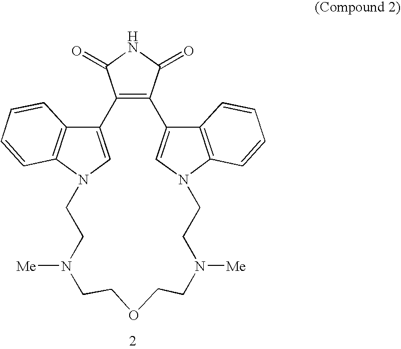

To a solution of Compound 1g (124 mg, 0.21 mmol) in DMF (18 mL) was added methyl-[2-(2-methylamino-ethoxy)-ethyl]-amine (144 mg, 1.09 mmol) and DIEA (169 mg, 1.30 mmol). The mixture was heated to 100° C. for 7 h, and then cooled to rt, extracted with EtOAc several times, washed with brine, dried (Na₂SO₄) and concentrated to give a red oil. The crude mixture was purified by flash chromatography (CH₂Cl₂/MeOH/NH₄OH 95/5/0.5) to give Compound 2 as an orange solid. ¹H NMR (300 MHz, CDCl₃) δ 7.33 (m, 5H), 7.22 (m, 3H), 7.01 (t, J=7.6 Hz, 2H), 4.17 (m, 4H), 3.27 (m, 4H), 2.82 (m, 4H), 2.37 (m, 10H). FAB-HRMS calcd for C₃₀H₃₃N₅O₃+H⁺, 512.2662, Found, 512.2651.

### EXAMPLE 3

### 10,11,12,13,14,16,17,18,19,20-decahydro-15-oxa-2,9,12,18,21-pentaaza-1H-diindolo[1,2,3-m,n:3′,2′,1′-r,s]cyclononadec-14,16,18-trien[16,17-c]-pyrrole-2,5-dione (Compound 3)

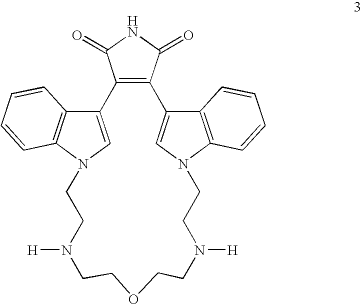

To a solution of Compound 1g (200 mg, 0.35 mmol) in DMF (15 mL) was added 2-(2-amino-ethoxy)-ethylamine (146 mg, 1.40 mmol) and DIEA (271 mg, 2.10 mmol). The mixture was heated to 100° C. for 7 h. The solvent was evaporated and the crude mixture was extracted with EtOAc several times. The combined extracts were washed with brine, dried (Na₂SO₄) and concentrated to give a red oil. The crude mixture was purified by flash chromatography to give Compound 3 as an orange solid. ¹H NMR (300 MHz, CDCl₃) δ 7.57 (s, 2H), 7.40 (d, J=8.2 Hz, 2H), 7.33 (d, J=8.0 Hz, 2H), 7.21 (d, J=7.2 Hz, 2H), 7.02 (t, J=7.3 Hz, 2H), 4.19 (t, J=4.9 Hz, 4H), 3.34 (t, J=5.0 Hz, 4H), 2.96 (t, J=4.9 Hz, 4H), 2.61 (t, J=4.9 Hz, 4H). FAB-HRMS calcd for C₂₈H₂₉N₅O₃+H⁺, 484.2349, Found: 484.2370.

### EXAMPLE 4

### 18-methyl-10,11,12,13,14,16,17,18,19,20-decahydro-15-oxa-2,9,12,18,21-pentaaza-1H-diindolo[1,2,3-m,n:3′,2′,1′-r,s]cyclononadec-14,16,18-trien[16,17-c]-pyrrole-2,5-dione

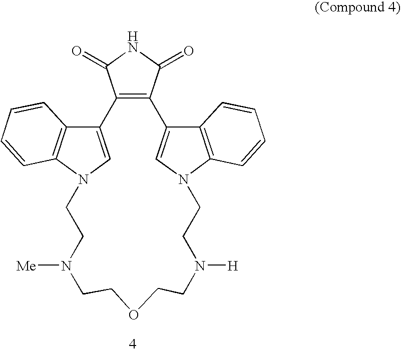

To a solution of Compound 3 (25.8 mg, 0.053 mmol) and DIEA (0.1 mL) in THF-DMF (5 mL: 0.5 mL) was added 160 μL (0.08 mmol) of 0.5 M CH₃I in THF. The mixture was stirred at r.t. for 1 h at which time most of starting material was still present. An additional 960 μL (0.48 mmol) of 0.5 M CH₃I in THF was added periodically over 4 h while strring at r.t. The solvent was then evaporated and the residue was dissolved in EtOAc. The solution was washed with H₂O, brine, and dried with Na₂SO₄, then concentrated to give the crude product. Preparative TLC separation (CH₂Cl₂/MeOH/NH₄OH 95/5/0.5) of the crude product afforded Compound 4, along with dimethylated Compound 2 and recovered Compound 3.

For Compound 4: ¹H NMR (500 MHz, CDCl₃) δ 7.55 (m, 2H), 7.43 (m, 1H), 7.34 (m, 1H), 7.31 (m, 1H), 7.22 (m, 2H), 7.12 (m, 2H), 6.96 (t, J=7.5 Hz, 1H), 4.28, (m, 2H), 4.05 (m, 2H), 3.29 (m, 4H), 3.05 (m, 2H), 2.60 (m, 4H), 2.31 (m, 2H), 2.12 (s, 3H). MS (ES) m/z 498.0 (M+H⁺).

### EXAMPLE 5

As a specific embodiment of an oral composition, 100 mg of Compound 1 is formulated with a sufficiently finely divided lactose to provide a total amount of 580 to 590 mg to fill a size 0 hard gel capsule.

Biological Examples

The compounds of the present invention were tested for biological activity in the following in-vitro and in-vivo methods.

### EXAMPLE 1

Protein Kinase C Histone-Based Assay

Compounds were evaluated for PKC selectivity using histone III as the substrate. The PKC isoforms α, β-II or γ were added to a reaction mixture containing 20 mM HEPES, (pH 7.4), 940 M CaCl₂, 10 mM MgCl₂, 1 mM EGTA. 100 μg/mL phosphatidylserine, 20 μg/mL diacylglycerol, 30 μM ATP, 1 μCi (³³P)ATP, and 200 μg/mL histone II. The reaction was incubated for 10 min at 30° C. Reactions were terminated by TCA precipitation and spotting on Whatman P81 filters. Filters were washed in 75 mM phosphoric acid and the radioactivity quantified by liquid scintillation counting.

Table 1 shows the biological activity in the histone-based assay as IC₅₀ values (μM) for representative compounds of the present invention.

TABLE 1
PKC Activity (IC₅₀ μM, Histone Based Assay)

| Cpd | Alpha | Beta I | Beta II | Gamma |
|---|---|---|---|---|
| 1 | 0.16 | 0.009 | 0.006 | 0.36 |
| 2 | 0.49 | 0.027 | 0.041 | 1.56 |
| 3 | 0.33 | 0.009 | 0.013 | 5.84 |
| 4 | 0.28 | 0.006 | 0.008 | 1.20 |

### EXAMPLE 2

Glycogen Synthase Kinase-3 Assay

Compounds were tested for the ability to inhibit recombinant rabbit GSK-3β protein using the following protocol. The test compound was added to a reaction mixture containing Protein phosphatase inhibitor-2 (PPI-2) (Calbiochem) (45 ng), rabbit GSK-3β protein (New England Biolabs) (0.75 units) and ³³P-ATP (1 μCi) in 50 mM Tris-HCl (pH 8.0), 10 mM MgCl₂, 0.1% BSA, 1 mM DTT and 100 μM Sodium Vanadate. The mixture was reacted for 90 minutes at 30° C. to allow phosphorylation of the PPI-2 protein and then the protein in the reaction was precipitated using 10% TCA. The precipitated protein was collected on filter plates (MultiScreen-DV/Millipore), which were subsequently washed. Finally, the radioactivity was quantified using a TopCount Scintillation Counter (Packard). GSK-3 inhibitory compounds resulted in less phosphorylated PPI-2 and thus a lower radioactive signal in the precipitated protein. Staurosporine or Valproate, known inhibitors of GSK-3β, were used as a positive control for screening.

Table 2 shows the biological activity in the GSK-3β assay as IC₅₀ values (μM) for representative compounds of the present invention.

TABLE 2
GSK-3β Assay Activity (IC₅₀ μM)

| Cpd | GSK-3β |
|---|---|
| 1 | 0.005 |
| 2 | 0.006 |
| 3 | 0.007 |

### EXAMPLE 3

Murine Retinopathy In-Vivo Model

The methods used in this model are known to those skilled in the art and are referenced in the scientific literature, such as Smith, L. E., Wesolowski, E., McLellan, A., Kostyk, S. K., D'Amato, R., Sullivan, R., and D'Amore, P. A. “Oxygen-induced retinopathy in the mouse”. Invest. Ophthalmol. Vis. Sci., 1994, January; 35(1): 101-11.

Compound 3 inhibited retinal neovascularization in the murine retinopathy model at 60 mg/kg.

It is to be understood that the preceding description of the invention and various examples thereof have emphasized certain aspects. Numerous other equivalents not specifically elaborated on or discussed may nevertheless fall within the spirit and scope of the present invention or the following claims and are intended to be included.

What is claimed is:

1. A compound of Formula (I): 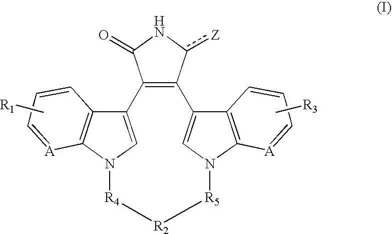
wherein
A is CH or N, whereby the A-containing ring system of Formula (I) thus forms 1H-indole or 1H-pyrrolo[2,3-b]pyridine, respectively;
Z is O, OH, or H, H;
R₁ and R₃ are independently selected from the group consisting of hydrogen, C₁₋₄alkyl, C₂₋₄alkenyl, C₂₋₄alkynyl, C₁₋₄alkoxy, C₁₋₄alkylthio, halogen, trifluoromethyl, trifluoromethoxy, hydroxy, hydroxy(C₁₋₄)alkyl, cyano, nitro, amino, and amino(C₁₋₄)alkyl; wherein amino and the amino portion of amino(C₁₋₄)alkyl are optionally and independently substituted with one to two C₁₋₄alkyl substituents;
R₄ and R₅ are independently C₂₋₈alkylene optionally substituted with oxo;
R₂ is —N(Rₐ)—C₁₋₄alkyl-X—C₁₋₄alkyl-N(R_{b}), wherein C₁₋₄alkyl is optionally substituted with one to four substituents independently selected from the group consisting of C₁₋₄alkyl, C₂₋₄alkenyl, C₂₋₄alkynyl, C₁₋₄alkoxy, C₁₋₄alkoxy(C₁₋₄)alkyl, carboxyl, carboxyl(C₁₋₄)alkyl, C₁₋₄alkoxycarbonyl, C₁₋₄alkoxycarbonyl(C₁₋₄)alkyl, amino, amino(C₁₋₄)alkyl, halogen, (halo)₁₋₃(C₁₋₄)alkyl, (halo)₁₋₃(C₁₋₄)alkoxy, hydroxy, hydroxy(C₁₋₄)alkyl, and oxo; wherein amino and the amino portion of amino(C₁₋₄)alkyl are optionally and independently substituted with one to two C₁₋₄alkyl substituents;
Rₐ and R_{b} are independently selected from the group consisting of hydrogen, C₁₋₆alkyl, C₂₋₆alkenyl, and C₂₋₆alkynyl; wherein C₁₋₆alkyl, C₂₋₆alkenyl, and C₂₋₆alkynyl are optionally substituted with fluoro;
X is O or S;
such that the —R₄—R₂—R₅— containing macrocycle does not exceed 25 atoms in size;
and enantiomers, diastereomers, racemates, and pharmaceutically acceptable salts thereof.

2. The compound according to claim 1 wherein A is CH, whereby the A-containing ring system of Formula (I) thus forms 1H-indole.

3. The compound according to claim 1 wherein Z is O.

4. The compound according to claim 1 wherein R₁ and R₃ are independently selected from the group consisting of hydrogen, methyl, methoxy, halogen, and hydroxy.

5. The compound according to claim 4 wherein R₁ and R₃ are each hydrogen.

6. The compound according to claim 1 wherein R₄ and R₅ are each C₂₋₄alkylene.

7. The compound according to claim 6 wherein R₄ and R₅ are each ethylene.

8. The compound according to claim 1 wherein R₂ is —N(Rₐ)—C₁₋₄alkyl-X—C₁₋₄alkyl-N(R_{b}).

9. The compound according to claim 8 wherein R₂ is —N(Rₐ)—CH₂CH₂—X—CH₂CH₂—N(R_{b}).

10. The compound according to claim 1 wherein Rₐ and R_{b} are independently hydrogen or C₁₋₆alkyl.

11. The compound according to claim 10 wherein Rₐ and R_{b} are independently hydrogen or methyl.

12. The compound according to claim 1 wherein X is S.

13. The compound according to claim 1 wherein X is O.

14. A compound of Formula (Ia): 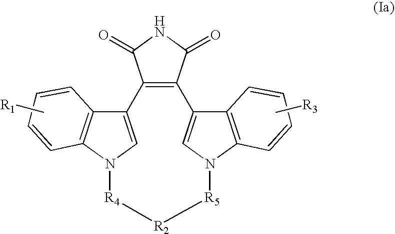
wherein
R₁ and R₃ are independently selected from the group consisting of hydrogen, C₁₋₄alkyl, C₂₋₄alkenyl, C₂₋₄alkynyl, C₁₋₄alkoxy, C₁₋₄alkylthio, halogen, trifluoromethyl, trifluoromethoxy, hydroxy, hydroxy(C₁₋₄)alkyl, cyano, nitro, amino, and amino(C₁₋₄)alkyl; wherein amino and the amino portion of amino(C₁₋₄)alkyl are optionally and independently substituted with one to two C₁₋₄alkyl substituents;
R₄ and R₅ are independently C₂₋₈ alkylene optionally substituted with oxo;
R₂ is —N(Rₐ)—C₁₋₄alkyl-X—C₁₋₄alkyl-N(R_{b}), wherein C₁₋₄alkyl is optionally substituted with one to four substituents independently selected from the group consisting of C₁₋₄alkyl, C₂₋₄alkenyl, C₂₋₄alkynyl, C₁₋₄alkoxy, C₁₋₄alkoxy(C₁₋₄)alkyl, carboxyl, carboxyl(C₁₋₄)alkyl, C₁₋₄alkoxycarbonyl, C₁₋₄alkoxycarbonyl(C₁₋₄)alkyl, amino, amino(C₁₋₄)alkyl, halogen, (halo)₁₋₃(C₁₋₄)alkyl, (halo)₁₋₃(C₁₋₄)alkoxy, hydroxy, hydroxy(C₁₋₄)alkyl, and oxo; wherein amino and the amino portion of amino(C₁₋₄)alkyl are optionally and independently substituted with one to two C₁₋₄alkyl substituents;
Rₐ and R_{b} are independently selected from the group consisting of hydrogen, C₁₋₆alkyl, C₂₋₆alkenyl, and C₂₋₆alkynyl; wherein C₁₋₆alkyl, C₂-₆alkenyl, and C₂₋₆alkynyl are optionally substituted with fluoro;
X is O or S;
such that the —R₄—R₂—R₅— containing macrocycle does not exceed 25 atoms in size;
and enantiomers, diastereomers, racemates, and pharmaceutically acceptable salts thereof.

15. The compound according to claim 14 wherein:
R₁ and R₃ are independently selected from the group consisting of hydrogen, C₁₋₄alkyl, C₁₋₄alkoxy, halogen, and hydroxy;
R₄ and R₅ are each C₂₋₄alkylene;
R₂ is —N(Rₐ)—C₁₋₄alkyl-X—C₁₋₄alkyl-N(R_{b})—; and,
Rₐ and R_{b} are independently hydrogen or C₁₋₄alkyl.

16. The compound according to claim 14 wherein Rₐ and R_{b} are independently hydrogen or methyl and R₁ and R₃ are independently selected from the group consisting of hydrogen, methyl, methoxy, halogen, and hydroxy.

17. The compound according to claim 14 wherein R₁ and R₃ are each hydrogen; R₄ and R₅ are each ethylene; R₂ is —N(Rₐ)—CH₂CH₂—X—CH₂CH₂—N(R_{b}); and Rₐ and R_{b} are independently hydrogen or methyl.

18. A compound of Formula (Ib): 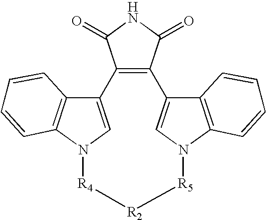
wherein
R₄ and R₅ are independently C₂₋₈alkylene optionally substituted with oxo;
R₂ is —N(Rₐ)—C₁₋₄alkyl-X—C₁₋₄alkyl-N(R_{b})—, wherein C₁₋₄alkyl is optionally substituted with one to four substituents independently selected from the group consisting of C₁₋₄alkyl, C₂₋₄alkenyl, C₂₋₄alkynyl, C₁₋₄alkoxy, C₁₋₄alkoxy(C₁₋₄)alkyl, carboxyl, carboxyl(C₁₋₄)alkyl, C₁₋₄alkoxycarbonyl, C₁₋₄alkoxycarbonyl(C₁₋₄)alkyl, amino, amino(C₁₋₄)alkyl, halogen, (halo)₁₋₃(C₁₋₄)alkyl, (halo)₁₋₃(C₁₋₄)alkoxy, hydroxy, hydroxy(C₁₋₄)alkyl, and oxo; wherein amino and the amino portion of amino(C₁₋₄)alkyl are optionally and independently substituted with one to two C₁₋₄alkyl substituents;
Rₐ and R_{b} are independently selected from the group consisting of hydrogen, C₁₋₆alkyl, C₂₋₆alkenyl, and C₂₋₆alkynyl; wherein C₁₋₆alkyl, C₂₋₆alkenyl, and C₂₋₆alkynyl are optionally substituted with fluoro;
X is O or S;
such that the —R₄—R₂—R₅— containing macrocycle does not exceed 25 atoms in size;
and enantiomers, diastereomers, racemates, and pharmaceutically acceptable salts thereof.

19. The compound according to claim 18 wherein R₄ and R₅ are each C₂₋₄alkylene; and, Rₐ and R_{b} are independently hydrogen or C₁₋₄alkyl.

20. The compound according to claim 18 wherein R₄ and R₅ are each ethylene; R₂ is-N(Rₐ)—CH₂CH₂—X—CH₂CH₂—N(R_{b})—; and, Rₐ and R_{b} are independently hydrogen or methyl.

21. A compound selected from the group consisting of:

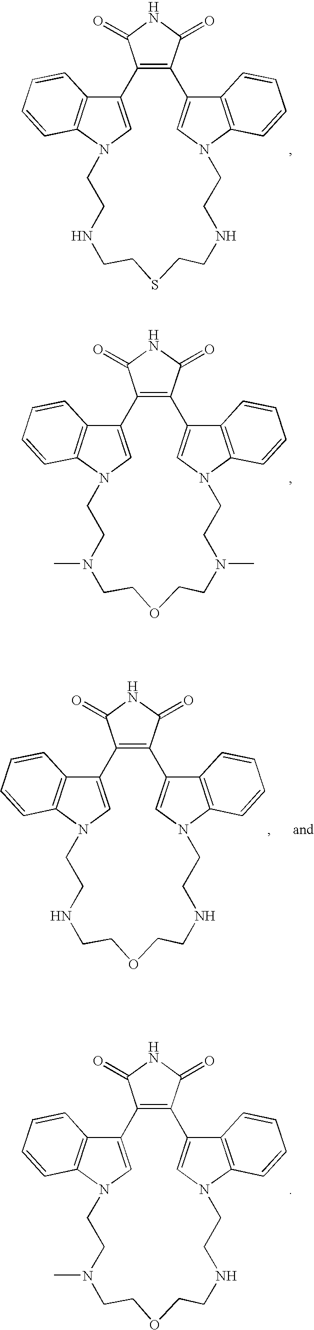

22. A compound selected from the group consisting of:
10,11,12,13,14,16,17,18,19,20-decahydro-15-thio-2,9,12,18,21-pentaaza-1H-diindolo[1,2,3-m,n:3′,2′,1′-r,s]cyclononadec-14,16,18-trien[16,17-c]-pyrrole-2,5-dione,
12,18-dimethyl-10,11,12,13,14,16,17,18,19,20-decahydro-15-oxa-2,9,12,18,21-pentaaza-1H-diindolo[1,2,3-m,n:3′,2′,1′-r,s]cyclononadec-14,16,18-trien[16,17-c]-pyrrole-2,5-dione,
10,11,12,13,14,16,17,18,19,20-decahydro-15-oxa-2,9,12,18,21-pentaaza-1H-diindolo[1,2,3-m,n:3′,2′,1′-r,s]cyclononadec-14,16,18-trien[16,17-c]-pyrrole-2,5-dione, and
18-methyl-10,11,12,13,14,16,17,18,19,20-decahydro-15-oxa-2,9,12,18,21-pentaaza-1H-diindolo[1,2,3-m,n:3′,2′,1′-r,s]cyclononadec-14,16,18-trien[16,17-c]-pyrrole-2,5-dione.

23. A pharmaceutical composition comprising a compound according to claim 1 and a pharmaceutically acceptable carrier.

24. A method for treating a kinase or dual kinase-mediated disease, condition or disorder in a subject in need thereof comprising the step of administering to the subject a therapeutically effective amount of the compound of claim 1.

25. The method according to claim 24 wherein the kinases are selected from the group consisting of protein kinase C α, protein kinase C β, protein kinase C γ and glycogen synthase kinase-3β.

26. The method of claim 24 wherein said therapeutically effective amount comprises a dose range of from about 0.001 mg/kg/day to about 300 mg/kg/day.

27. The method of claim 24 wherein the disease, condition or disorder is selected from the group consisting of cardiovascular diseases, diabetes, diabetes-associated disorders, inflammatory diseases, immunological disorders, dermatological disorders, oncological disorders and CNS disorders, comprising administering to a subject in need of such treatment a therapeutically effective amount of a compound of claim 1.

28. The method of claim 27 wherein cardiovascular diseases are selected from acute stroke, heart failure, cardiovascular ischemia and impaired cardiac function following ischemia, thrombosis, atherosclerosis, hypertension, restenosis, retinopathy of prematurity or age-related macular degeneration.

29. The method of claim 27 wherein diabetes is selected from insulin dependent diabetes or Type II non-insulin dependent diabetes mellitus.

30. The method of claim 27 wherein diabetes-associated disorders are selected from impaired glucose tolerance, insulin signaling defects, insulin resistance, metabolic syndrome X, diabetic retinopathy, proliferative retinopathy, retinal vein occlusion, macular edema, cardiac hypertrophy associated with heart failure, cardiomyopathy, nephropathy or neuropathy.

31. The method of claim 27 wherein inflammatory diseases are selected from neutrophil and cytokine migration, bone marrow degranulation, vascular permeability, inflammation, asthma, rheumatoid arthritis or osteoarthritis.

32. The method of claim 27 wherein immunological disorders are selected from transplant tissue rejection, HIV-1 transcription and viral replication or immunological disorders treated or ameliorated by PKC modulation.

33. The method of claim 27 wherein dermatological disorders are selected from psoriasis, hair loss or baldness.

34. The method of claim 27 wherein oncological disorders are selected from cancer or tumor growth and other diseases associated with uncontrolled cell proliferation such as recurring benign tumors as well as including proliferative angiopathy and angiogenesis.

35. The method of claim 34 wherein cancer or tumor growth is selected from breast, brain, kidney, bladder, ovarian or colon cancer or lymphocytic leukemia.

36. Use of the compound of claim 1 as an adjunct to chemotherapy and radiation therapy.

37. The method of claim 27 wherein CNS disorders are selected from chronic pain, neuropathic pain, epilepsy, chronic neurodegenerative conditions, mood disorders and ischemia-related diseases.

38. The method of claim 37 wherein chronic neurodegenerative conditions are selected from dementia or Alzheimer's disease.

39. The method of claim 37 wherein mood disorders are selected from schizophrenia, manic depression or neurotraumatic, cognitive decline.

40. The method of claim 37 wherein ischemia-related diseases are selected from diseases resulting from head trauma, such as from acute ischemic stroke, injury or surgery or from transient ischemic stroke, such as from coronary bypass surgery or other transient ischemic conditions.

41. The method of claim 24 wherein the disease, condition or disorder is selected from the group consisting of treating or ameliorating diabetes or Alzheimer's disease.

42. A pharmaceutical composition made by mixing a compound of claim 1 and a pharmaceutically acceptable carrier.

43. A process for making a pharmaceutical composition comprising mixing a compound of claim 1 and a pharmaceutical acceptable carrier.

44. Use of the compound of claim 1 in the manufacture of a medicament for treating or ameliorating a kinase or dual-kinase mediated disease, condition or disorder.
